(19) United States
(12) Patent Application Publication
(10) Pub. No.: US 2009/0121142 A1
(43) Pub. Date: May 14, 2009

(54) Radiation detector module, radiation detector and imaging tomography device

(76) Inventors: Bjorn Heismann, Erlangen (DE); Debora Henseler, Erlangen (DE)

Correspondence Address: HARNESS, DICKEY & PIERCE, P.L.C., P.O.BOX 8910, RESTON, VA, 20195, US

(21) Appl. No.: 12/219,274

(22) Filed: Jul. 18, 2008

(30) Foreign Application Priority Data
Jul. 20, 2007 (DE) 10 2007 033 875.0

(51) Int. Cl. G01T 1/166; G01T 1/20; A61B 6/03

(52) U.S. Cl. 250/363.04; 250/361 R; 378/4; 250/362

(57) ABSTRACT

An embodiment of the invention relates, in particular, to a radiation detector module for producing a radiation detector for computed tomography, having a first operating mode for quantitative and/or energy-selective detection of x-radiation. An embodiment relates to a radiation detector module including a scintillation layer for converting the x-radiation into light, and a photodetection unit for detecting the light, the photodetection unit including a multiplicity of silicon photomultipliers.

### PRIORITY STATEMENT

The present application hereby claims priority under 35 U.S.C. §119 on German patent application number DE 10 2007 033 875.0 filed Jul. 20, 2007, the entire contents of which is hereby incorporated herein by reference.

### FIELD

Embodiments of the invention generally relate to a radiation detector module, a radiation detector including a number of radiation detector modules, and/or an imaging tomography device including the radiation detector.

### BACKGROUND

In order to detect x-radiation in computed tomography, so-called integrating radiation detectors, inter alia, are used in accordance with the prior art. Such radiation detectors typically comprise a number of scintillation elements for converting x-ray quanta into light, that is to say into photons. Photodiodes for converting the photons into electrical signals are arranged on the scintillation elements. The electrical signals are read out by means of integrating read out electronics in a prescribed signal/read out cycle, and subsequently digitized. For each signal/read out cycle; the signal read out is approximately proportional to the number of photons produced, and to the average energy of the x-ray quanta responsible therefor. Reference may be made to U.S. Pat. No. 6,630,675 by way of example in relation to the prior art of such scintillator photodiode systems.

According to the prior art, in addition to the integrating radiation detectors so-called counting radiation detectors are also used in computed tomography. Because of the comparatively high flux rates occurring in computed tomography, which are in the region of 10⁹/s·mm², there is a need for radiation detectors that are capable of detecting such flux rates. Direct converters produced from semiconductor materials such as CdZnTe, for example, have proved to be particularly suitable for this according to the prior art.

In the case of such direct converters, the x-ray quanta absorbed in the semiconductor material are directly converted in a single conversion step into electric charges that influence electrical signals on appropriately provided electrodes. The number and energy of the x-ray quanta can be determined on the basis of the electrical signals, that is to say the x-radiation can be detected quantitatively and energy-selectively. Reference may be made to U.S. Pat. No. 7,139,362, for example, in relation to the prior art of such direct converter systems.

A disadvantage of the first named scintillator photodiode systems resides in that these are not fast enough for the quantitative and energy-selective detection of x-radiation. Their application to the integrating detection of x-radiation is correspondingly limited.

Disadvantages of the second named direct converter systems, compared with the scintillator photodiode systems, reside, inter alia, in higher material costs and a more complicated construction technique, and higher production costs associated therewith. Apart from that, the semiconductor materials suitable for direct converter systems, such as CdZnTe, are generally less environmentally compatible, for example toxic.

### SUMMARY

In at least one embodiment of the invention, at least one disadvantage according to the prior art is aimed at being eliminated, or at least reduced. One aim in at least one embodiment, in particular, is to provide a scintillator photodetection-based radiation detector module and a corresponding radiation detector for x-ray computed tomography, which module and detector permit a quantitative and/or energy-selective detection of x-radiation. A further aim of at least one embodiment is to provide an imaging tomography device in the case of which a scintillator photodetection-based radiation detector performs a quantitative and/or energy-selective detection of x-radiation.

A first aspect of at least one embodiment of the invention relates to a radiation detector module for producing a radiation detector for x-ray computed tomography. Provided in the case of the radiation detector is a first operating mode that permits a quantitative and/or energy-selective detection of x-radiation. This means that x-ray quanta of the x-radiation can be detected according to their number and/or energy.

To this end, the radiation detector includes a scintillation layer, produced from a scintillation material, for converting the x-radiation into light, and a photodetection unit, coupled to the scintillation layer, for detecting the light. The coupling can consist, for example, in that the photodetection unit is fitted with a light entry side on a light exit side of the scintillation layer.

Unless something to the contrary is mentioned, within the concept of wave/particle dualism of physics the terms radiations and quanta or photons are used as being of equal status one to the other within the scope of this invention. That is to say, x-ray or gamma radiation has equal status with x-ray or gamma quanta; light is of the same status as photons. However, this is not intended to mean that the fundamentally different nature of radiation and quantum is of no importance to the invention.

According to at least one embodiment of the invention, the photodetection unit provided for detecting the light includes a number, or else a multiplicity, of silicon photomultipliers that are also denoted for short as SiPM. The arrangement of the SiPMs is essentially arbitrary and can, in particular, be in matrix fashion.

In simplified terms, an SiPM includes a matrix of photodiodes connected in parallel and operated in Geiger mode, in particular avalanche photodiodes. Within the scope of at least one embodiment of the invention, the individual parallel-connected photodiodes, including in each case further electronic components that may be provided, are denoted as detection cells. To this extent, the photodetection unit comprises a number or a multiplicity of SiPMs that in turn have a number or a multiplicity of detection cells.

Operation in Geiger mode means that the photodiodes are driven with a bias voltage above the breakdown voltage. In Geiger mode, a photon detected by a detection cell can activate a charge carrier avalanche with high gain. Within the scope of at least one embodiment of the invention, the activation of a detection cell is also denoted as triggering. Consequently, a photon that is suitable for activating a detection cell is also denoted as a triggerable photon, and an activated detection cell is denoted as triggered. Words belonging to the “triggering” word family are used in a corresponding sense.

The detection cells can be assigned photon counting properties on the basis of the specific method of functioning. For the sake of completeness, it may be mentioned that each detection cell additionally has a quenching resistor connected in series with the photodiode. The quenching resistor is important for resetting a triggered detection cell into a triggerable state, or in other words for resetting from a detection-passive state into a detection-active state. To clarify terminology, it may be added that the detection-passive state includes the period from triggering up to resetting into the triggerable, that is to say detection-active, state.

The avalanche photodiodes supply a signal that is proportional to the number of the activated charge carrier avalanches. This means that the signal is proportional to the number of triggered (avalanche) photodiodes. Because of the parallel connection of the detection cells, the SiPM acts as a proportional counter. That is to say, the output signal of the SiPM is approximately proportional to the number of the triggered detection cells, at least for a specific period. However, this proportionality presupposes that the SiPM is not yet in saturation. A non-saturated state means in this case that the number of the detection cells in the detection-passive state is substantially smaller than the number of the detection-active, that is to say triggerable, detection cells.

In addition, and by way of example, reference may be made to the following as regards the method of functioning of the SiPMs:
Otte et al.: “Status of Silicon Photomultiplier Developments as optical Sensors for MAGIC/EUSO-like Detectors”, in 29th International Cosmic Ray Conference Pune (2005) 00, 101-106;
Sadygov et al.: “Three advanced designs of micro-pixel avalanche photodiodes: Their present status, maximum possibilities and limitations”, in Nuclear Instruments and Methods in Physics Research A 567 (2006), 70-73; and
a product description for the Hamamatsu company: “MPPC, Multi-Pixel Photon Counter”, available at the URL http://sales.hamamatsu.com/assets/pdf/catsandguides/mppc_kapd0002e01.pdf.

It follows from the abovenamed references to the literature that an SiPM is capable of quantitatively detecting small photon fluxes with high accuracy. Consequently, the SiPM technology is particularly suitable for positron emission tomography (PET) or single photon emission computed tomography (SPECT), since there is a need here to detect only comparatively low counting rates in the region of a few hundred events/s·mm². By way of comparison: as already mentioned, counting rates are in the region of 109/s·mm² in the case of x-ray computed tomography.

Proceeding therefrom, the SiPM technology seems to be unsuitable with regard to the counting rates prevailing in x-ray computed tomography. However, contrary to this appearance the inventors have discovered that the SiPM technology is suitable even for radiation detectors for quantitative and/or energy-selective detection of x-radiation in x-ray computed tomography.

In quantitative terms, what is meant here is that the number of the x-ray quanta absorbed by the radiation detector can be determined. The latter is also known by the keyword “counting radiation detector”: Energy-selective is intended to mean that the energy of the x-ray quanta can be determined, at least can be placed in a prescribed energy window, or can be classified with reference to a prescribed energy threshold.

Advantages of at least one embodiment of the inventive radiation detector based on scintillator SiPM technology as against conventional direct converter detectors reside in the fact that clear-cut cost advantages and an improved environmental compatibility can be achieved with regard to the materials used. It may be mentioned in this context that the SiPM is based on conventional, established silicon technology, whereas in the case of direct converter detectors it is necessary to have recourse to other materials, sometimes still in the development stage.

Furthermore, the constructional technique is simpler. This results, for example, in simple production processes and, attendant thereon, comparatively lower production costs for the inventive radiation detector module.

Aside from this, an optimum basis for further optimizations is offered by the technological maturity existing for conventional scintillator photodetection systems and advances as against direct converter systems. In particular, it is possible to have recourse to conventional materials, as a result of which known advantages can be used for dose usage and evaluation of spectral information.

A second aspect of at least one embodiment of the invention relates to a radiation detector having a number of radiation detector modules according to the first aspect of at least one embodiment of the invention.

A third aspect of at least one embodiment of the invention relates to an imaging tomography device, in particular an x-ray computed tomography device having a radiation detector according to the second aspect of at least one embodiment of the invention.

Further aspects of at least one embodiment of the invention relate to the use of the radiation detector according to the second aspect in an x-ray computed tomography device, a combined x-ray positron emission tomography device (PET/CT), or a combined x-ray single photon emission tomography device (SPECT/CT), and to the use of the radiation detector module according to the first aspect for the purpose of producing a radiation detector for the corresponding tomography devices.

Reference is made to designs relating to the radiation detector module according to the first aspect for advantages and advantageous effects of the second and third aspects and of the further aspects of at least one embodiment of the invention.

### BRIEF DESCRIPTION OF THE DRAWINGS

The invention is explained in more detail below with the aid of example embodiments and figures, of which:

FIG. 1 shows an x-ray computed tomography device having a radiation detector according to the second aspect of an embodiment of the invention with a number of radiation detector modules according to the first aspect of an embodiment of the invention;

FIG. 2 shows a cross section through a radiation detector module of FIG. 1;

FIG. 3 shows a schematic sketch of a design of a silicon photomultiplier, SiPM for short;

FIG. 4 shows a plan view of the radiation detector module illustrated in FIG. 1; and

FIG. 5 shows a schematic of a characteristic of an output signal of an SiPM in the case of immediately sequential triggering.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Identical or functionally identical elements are denoted throughout in the figures by identical reference numerals. The illustrations in the figures are schematic, given by way of example, and not true to scale. Scales can vary between the figures. The x-ray computed tomography device and the radiation detector will be examined below only to the extent necessary to understand embodiments of the invention.

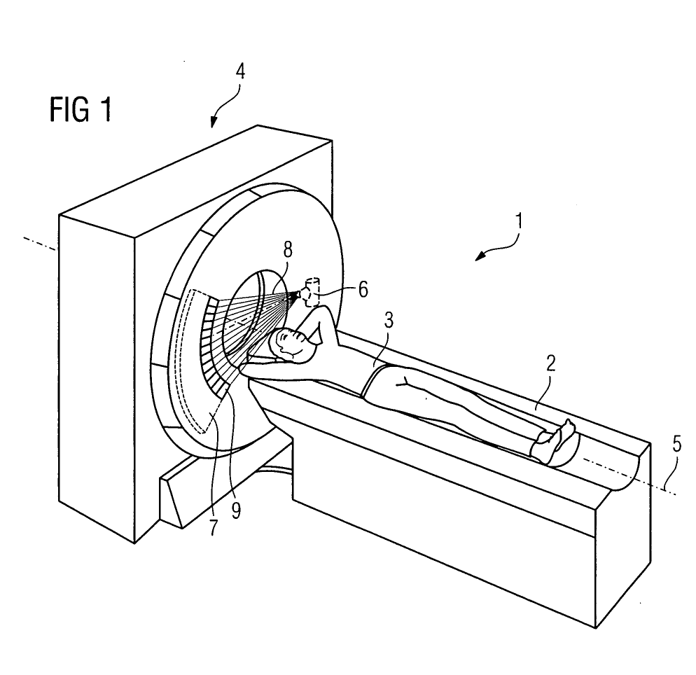

FIG. 1 shows an x-ray computed tomography device 1 according to the third aspect of an embodiment of the invention. The x-ray computed tomography device 1 includes a patient positioning table 2 for positioning a patient 3 to be examined. Furthermore, the x-ray computed tomography device 1 includes a gantry 4 in whose housing a tube detector system mounted rotatably about a system axis 5 is accommodated. The tube detector system includes an x-ray tube 6 and a radiation detector 7 arranged lying opposite thereto. During operation, x-radiation 8 emanates from the x-ray tube 6 in the direction of the radiation detector 7 and can be detected quantitatively and/or in an energy-selective fashion by means of the radiation detector 7. The radiation detector 7 has a number of radiation detector modules 9 according to the first aspect of an embodiment of the invention.

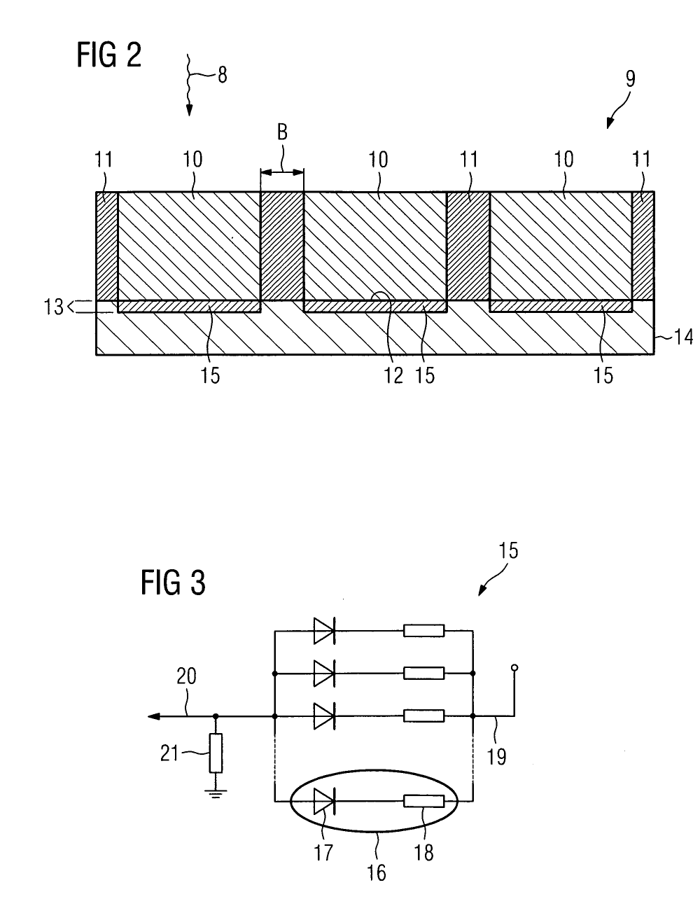

The radiation detector modules 9 are constructed using scintillator photodetection technology. FIG. 2 shows a cross section through such a radiation detector module 9. The radiation detector module 9 has a number of scintillation elements 10 that are arranged next to one another, are produced from a scintillation material, and are separated from one another by septa 11. The scintillation elements 10 convert the x-radiation 8 into light. More accurately, photons are produced by the interaction processes caused by x-radiation 8 or by x-ray quanta of the x-radiation 8.

The septa 11 are provided in order to avoid optical crosstalk of the scintillation elements 10, as a result of which it is possible to, inter alia, to improve the spatial resolution. Otherwise than with the pixelation effected by the septa 11, it is also possible to provide a continuous scintillation layer instead of the individual scintillation elements 10.

The number of scintillation elements 10 shown in FIG. 2 is merely to be understood by way of example, which means that it is also possible within the scope of an embodiment of the invention to arrange a larger or smaller number of scintillation elements 10 next to one another. A width B of the septa 11 is a function of the respective requirements and edge conditions. For example, widths B of between 50 μm and 350 μm, with particular preference of between 80 μm and 300 μm, come into consideration for x-ray computed tomography.

A photodetection unit 13 is fitted on a light exit side 12 on each scintillation element 10. The photodetection unit 13 can, in turn, be fitted on a substrate 14 or be designed in a fashion integrated with the substrate 14. The photodetection unit 13 comprises a multiplicity of so-called silicon photomultipliers 15, SiPM for short, or Geiger mode avalanche photodiodes. In the present example, each scintillation element 10 is assigned exactly one SiPM 15. Within the scope of an embodiment of the invention, it is also possible for a number of SiPM 15 to be assigned to a scintillation element 10, that is to say a SiPM 15 can comprise a number of sub-photodetection units. Correspondingly, a scintillation element 10 can also be subdivided into a number of sub-scintillation elements. These subdivisions can be substantially of any desired, fractal-like design. The number of the sub-photodetection units can correspond to the sub-scintillation elements, and each sub-scintillation element can be assigned exactly one sub-photodetection unit. The number of the sub-photodetection units or sub-scintillation elements can be given by N×N, in which N is a natural number. For example, N can be equal to 2, 4, 5 etc. Owing to the variability of the respective number, the radiation detector module can be adapted to the respectively desired resolution and flux rates of the x-ray quanta which, for example, opens up the possibility of improving the counting rate capacity of the radiation detector module.

Detection surfaces, that is to say detection cross sections, of the scintillation elements 10—and, correspondingly, of the SiPMs 15—substantially determine the spatial resolution of the radiation detector module 9. The sizes of the detection cross sections are limited, inter alia, by the respective state of technology for the production. According to the current state of technology, detection cross sections suitable for x-ray computed tomography can be implemented which are smaller than 15 mm², smaller that 10 mm², smaller than 5 mm², or even smaller than 1 mm². However, with regard to the technical trend of continuous miniaturization of the detection cross sections, much smaller values are conceivable, however, and are in no way opposed to the inventive thinking.

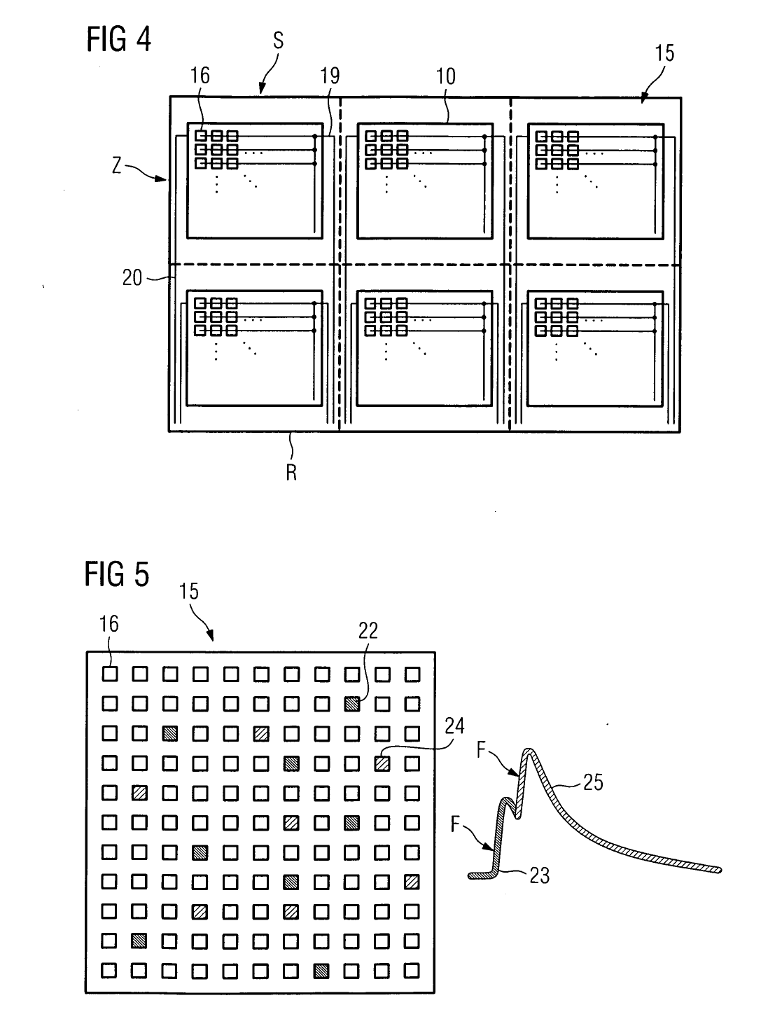

FIG. 3 shows a schematic sketch of a design of an SiPM 15. The SiPM 15 has a multiplicity of parallel-connected detection cells 16 that each comprise, in turn, a Geiger mode avalanche photodiode 17 and a quenching resistor 18. On the side of the quenching resistors 18, each detection cell 16 makes contact with or is connected to a so-called bias line 19. The bias lines 19 are further examined in more detail in FIG. 4. On the side of the photodiodes 17, each detection cell 16 makes contact with a signal line 20 that is connected to ground in this example via an output resistor 21. The contact making and reading out of the signal line 20 can alternatively also be configured otherwise than in this example.

FIG. 4 shows a plan view of the radiation detector module 9, the scintillation elements 10 being indicated merely by dash-dotted lines. As may be seen from FIG. 4, the SiPMs 15 are arranged in rows Z and columns S, the number of the rows Z and columns S being, in turn, exemplary and not to be viewed as limiting. The detection cells 16 of each SiPM 15 are likewise arranged in matrix fashion in rows and columns. For the sake of clarity, in each case only a few of the detection cells 16 of the matrix-like arrangement are illustrated.

Bias contacts of the detection cells 16 in each case make contact with a single bias line 19 routed to an end face edge R of the radiation detector module 9. Signal contacts of the detection cells 16 in each case make contact with a single signal line 20 routed to the end face edge R of the radiation detector module 9. The line routing in and/or on the radiation detector module 9 can be substantially simplified by providing only in each case a single bias line 19 and/or signal line 20. However, it is also possible to make contact with the bias contacts and/or signal contacts via a respectively separately routed bias line 19 and/or signal line 20. As in the present example, the bias lines 19 and signal lines 20 are preferably routed, in interspaces between the SiPMs 15, to the edge R of the radiation detector module 9. In this way, the available space can be optimally utilized and a particularly compact design can be achieved.

In a departure from the routing of the bias lines 19 and signal lines 20 that is shown in FIG. 4, it is also possible that—with reference to the view shown in FIG. 3—the bias contacts and/or signal contacts are fitted on the underside of the SiPMs 15 and are connected there to bias lines and/or signal lines. This is advantageous for the line routing and contact making, particularly whenever it is not possible, or only conditionally possible, to route lines in the interspaces because of the spatial proximity of the SiPMs 15.

Further ways of making contact with the bias contacts and signal contacts are also conceivable. For example, mention may be made of contact being made in lines or rows between the detection cells 16 and in each case a single bias line 19 and/or signal line 20.

The signal lines 20 are connected to electronic components and/or circuits (not shown), for example ASICs (Application Specific Integrated Circuits) in order to process respective output signals of SiPMs 15 and/or detection cells 16. The components and/or circuits can be provided or formed on the substrate 14 or in an integral fashion therewith, in order to avoid or reduce disturbing influences and for the purpose of particularly compact design. The circuits can be, in particular, CMOS structures (CMOS: Complementary Metal Oxide Semiconductor).

There follows a more detailed discussion of preconditions for the quantitative and/or energy-selective detection of the x-radiation 8 by means of the radiation detector 9.

Use should be made of scintillation materials having a decay time that copes with the quantum flux rates so that it is possible to achieve a particularly accurate quantitative detection of the x-ray quanta in the case of the quantum flux rates in the region of 10⁹/s·mm² that are customary in x-ray computed tomography. In this case, the decay time should be of the order of magnitude of a few nanoseconds, ns for short, up to a few 10 ns. For example, the decay time can be smaller than 40 ns to 20 ns, smaller than 20 ns to 10 ns, or else smaller than 10 ns, in particular smaller than 5 ns. Here, the decay time is understood as the time in which the number of photons produced by a quantum absorption event has decayed to 1/e. The following may be named by way of example as suitable scintillation materials: Lu₂SiO₅: (Ce), LaBr₃: (Ce), YAP:Ce (YAP: Yttrium-Aluminum-Perowskit), Lu(Y)AP:Ce. Respective decay times of the abovementioned scintillation materials are approximately 40 ns, 16 ns to 26 ns and 27 ns. Of course, scintillation materials having more advantageous, in particular yet smaller, decay times also come into consideration.

Presently, typical pixel sizes on radiation detectors 7 in x-ray computed tomography are in the range of between 1 mm×1 mm to 1.5 mm×10 mm, depending on the spatial resolution that is respectively the goal. For example, and without restriction of generality, a basic size of 1 mm×1 mm is used below for the SiPMs 15 or scintillation elements 10, that is to say the detection cross section is approximately 1 mm². Furthermore, an x-ray quanta energy range of from 30 keV to 120 keV that is usual for x-ray computed tomography is adopted, with quantum flux rates in the region of 10⁹/s·mm².

In order to be able to achieve energy-selective detection of the x-radiation 8 that is as accurate as possible, it is, on the one hand, of particular importance that the number of detection cells 16 is sufficiently large that it is still possible also to detect high energy x-ray quanta that excite a comparatively high number of photons, without SiPMs 15 operating in the region of saturation. Here, operating saturation is intended to mean that a substantial proportion of the photodiodes 17 is in the detection-passive state, on average. On the other hand, the SiPMs 15 should have a photon detection efficiency, PDE for short, that is sufficient still to be able reliably and accurately to detect even low energy x-ray quanta that excite a comparatively low number of photons. This requires, in particular, that the output signals, caused by the low energy x-ray quanta, of the SiPMs 15 are not superposed too excessively by quantum noise. The PDE can be described by the probability that an incident photon leads to the triggering of a detection cell 16.

For the quantum flux rates usual in x-ray computed tomography, the decay time of the output signal of each SiPM 15 should lie in the region of a few nanoseconds, preferably fewer than 3 ns, preferably fewer than 1 ns. For the same reason, a recharging time of each detection cell 16 should be less than 10 ns, preferably less than 3 ns, preferably less than 1 ns. Here, recharging time is understood essentially as the period of the detection-passive state of the detection cell 16.

Other parameters of the SiPM 15 that determine the quantitative and/or energy-selective detection of the x-ray quanta are: the size of the detection cells 16, the number of the detection cells 16 per SiPM 15, or their areal density, an absorption coefficient for photons in an intermediate layer, and PDE. Typical sizes of the detection cells 16 lie in the range from 10 μm to 100 μm. The areal density can be a few hundred to a few thousand detection cells per mm² of radiation detector area. The last mentioned parameters are closely linked to a cell period, for example a pitch, of the detection cells 16. Cell periods suitable for x-ray computed tomography can preferably lie in the range of from 50 micrometers to 25 micrometers, from 25 micrometers to 20 micrometers, from 20 micrometers to 10 micrometers, or fewer than 10 micrometers. The number of the detection cells 16 per SiPMs advantageously lies in the present field of application between 1500 and 2500, between 2500 and 5000, between 5000 and 10 000, or is greater than 10 000.

It may be mentioned for the sake of completeness that the PDE results from the product of a geometric filling factor and quantum efficiency, with the SiPM still to operate in the region of saturation. The quantum efficiency is, in turn, a function of the absorption efficiency and triggering probability. Typical values of the PDE lie in the range of from 10% to 70%. It should be noted that high values of the PDE can be achieved only in the case of a comparatively large detection cross section of the detection cells 16, because the geometric filling factor acts in a limiting fashion in the case of small detection cross sections.

The table below compares properties of five example SiPMs 16 for selected values of pitch, number of detection cells 16 per SiPM 15, abbreviated as N_{ges}, and PDE.

The tables are based on the following assumptions:
1. The x-ray quanta have a minimum energy of 30 keV and a maximum energy of 120 keV, this corresponding to the situation in x-ray computed tomography.
2. 30 photons are produced per keV of x-radiation in the scintillation element 10. This means that a scintillation element 10 produces 900 photons by absorbing an x-ray quantum having an energy of 30 keV, and produces up to 3600 photons by absorbing an x-ray quantum having an energy of 120 keV.

| | #1 | | #2 | | #3 | | #4 | | #5 | |
|---|---|---|---|---|---|---|---|---|---|---|
| Pitch | 50 μm | | 25 μm | | 20 μm | | 10 μm | | 10 μm | |
| N_{ges} | 400 | | 1600 | | 2500 | | 10 000 | | 10 000 | |
| PDE | 50% | | 25% | | 20% | | 10% | | 25% | |
| E₀/keV | 30 | 120 | 30 | 120 | 30 | 120 | 30 | 120 | 30 | 120 |
| N_{TP} | 450 | 1800 | 225 | 900 | 180 | 720 | 90 | 360 | 225 | 900 |
| (ΔE/E₀)/% | 11.1 | 5.5 | 15.7 | 7.8 | 17.5 | 8.8 | 24.8 | 12.4 | 15.7 | 7.8 |
| N_{trig}/% | 67.5 | 98.9 | 13.1 | 43.0 | 6.9 | 25.0 | 0.9 | 3.5 | 2.2 | 8.6 |
| NL/% | 40.0 | 78.0 | 6.7 | 23.5 | 3.5 | 13.1 | 0.4 | 1.8 | 1.1 | 4.4 |

The following abbreviations are used in the table:
N_{ges}: Number of the detection cells 16 per SiPM 15;
E₀/keV: mean energy of the x-ray quanta;
N_{TP}: number of the triggerable photons; N_{TP} results from the product of the number of photons produced for the respective energy E₀, and the PDE;
ΔE/E₀: energy resolution of the SiPM 15 for the respective mean energy E₀, specified in %, a Poisson distribution of the energy about the mean energy E₀ being used as a basis;
N_{trig}: percentage proportion of those detection cells 16 that are triggered on average by the N_{TP}; and
NL: nonlinearity between N_{TP} and the number of the triggered detection cells 16, expressed in percent.

It is clear from the table that the energy resolution reduces for diminishing pitch and rising N_{ges}, that is to say for increasing sizes of the detection cells 16. The reason for this is, inter alia, that the PDE diminishes under these preconditions. At the lower end of the energy scale of the x-ray quanta, that is to say at 30 keV, the energy resolution is respectively worse than at the upper end of the energy scale—at 120 keV. The reason for this is that fewer photons are produced at the lower end and this—as conditioned by quantum statistics—leads to an increase in the half-value width ΔE. An energy resolution of 25% at the lower end of the energy scale is certainly sufficient for x-ray computed tomography applications. The reason for this is that in x-ray computed tomography it is still sufficient to have an energy discrimination with comparatively few energy stages or energy windows. Particularly advantageous values result for the example of #5 with a pitch of 10 μm and 25% PDE: the energy resolution at the lower end of the energy scale lies at a comparatively good value of 15.7%.

In order to be able to detect the x-radiation 8 as accurately as possible in terms of quantity and energy selectivity, the number of the detection cells 16 should be selected such that the nonlinearity NL is smaller than 20%, preferably smaller than 10%, particularly advantageously smaller than 5%. Taking account of the values for the energy resolution, example #5 proves, in turn, to be particularly advantageous.

N_{trig} and NL are interlinked. The more that N_{trig} approximates to the total number of the detection cells 16 of an SiPM 15, the greater is the nonlinearity NL. The reason for this is, inter alia, that at a specific instant a detection cell 16 can be triggered only once even when struck by a number of photons. This is associated with the fact that a detection cell 16 in the detection-passive state cannot be triggered again. Since it is certainly possible that the triggerable photons produced by an x-ray quantum strike a detection cell 16 in the detection-passive state, the number of the triggered detection cells 16 is on average smaller than N_{TP}.

The mean number N̅_{trig} of the detection cells N_{trig} triggered for a prescribed N_{TP} and prescribed number N_{ges} can be calculated as follows:

N̅_{trig}=N_{ges}·(1−Exp(−N_{TP}/N_{ges}))

in which case it holds that: N_{trig}= N̅_{trig}/N_{ges}.

When N_{TP} is of the order of magnitude of the triggerable detection cells 16, saturation effects occur that are reflected in higher nonlinearities NLs. Although the nonlinearities NLs can be corrected, they should be avoided, especially in the high energy region of the x-ray quanta, since this leads to an impairment of the energy-selective detection of the x-ray quanta. However, such disadvantageous effects can be avoided in a particularly advantageous way when N_{ges}, preferably in the entire energy spectrum of the x-radiation 8, is larger than N_{TP} by a factor of approximately two. However, it is particularly advantageous to operate far removed from saturation when, for example, N_{ges} is larger than N_{TP} by a factor of more than ten. The latter case is explained in greater detail by way of example with the aid of FIG. 5.

FIG. 5 shows a schematic of a profile of an output signal of an individual SiPM 15 in the case of sequential absorption of two x-ray quanta. Illustrated on the left in FIG. 5 is a plan view of the SiPM 15 with detection cells 16 arranged in matrix fashion. A first group 22, here marked with close hatching, of detection cells 16 is triggered by photons produced during absorption of a first x-ray quantum in the associated scintillation element 10 (not shown). The charge carrier avalanches caused by the detection cells 16 of the first group 22 lead to a first output signal component 23 illustrated on the right schematically and correspondingly in close hatching. Photons produced during absorption, following immediately thereafter, of a second x-ray quantum in the scintillation element 10 trigger a second group 24 of detection cells 16, here marked with loose hatching, while the first group 22 of detection cells 16 is still in the detection-passive state. The charge carrier avalanches caused by the detection cells 16 of the second group 24 lead to a second output signal component 25 that is represented schematically on the right and correspondingly in loose hatching, and is superposed on the first output signal component 23. The first output signal component 23 and the second output signal component 25 can be identified, for example, by way of pulse height discrimination and other methods, so that a quantitative, energy-selective detection of the first and second x-ray quantum is possible despite superposition.

A further improvement in the quantitative detection of the x-radiation, in particular in the counting rate capability, that is to say the maximum possible counting rate or the maximum possible quantitatively detectable quantum flux rate, can be achieved when it is not the total output signal that is evaluated for the individual absorbed x-ray quanta, but only the rising edge F of the respective output signal component 23 and 25, respectively, for example as counting pulse. Owing to the relatively steep rise in the edges F of the output signal components 23 and 25, the x-ray quanta can be detected sufficiently accurately even in the case of high flux rates despite the superpositions, which are also denoted as “pile-up”.

Despite triggering of the first group 22 of detection cells 16, that is to say despite the fact that the detection cells 16 of the first group 22 are in the detection-passive state, it is evident that there are still sufficient other detection cells 16 available to be able to detect the subsequent second x-ray quantum with satisfactory accuracy. This means that saturation-induced falsifications of the output signals can be avoided by means of a high number of detection cells 16 per SiPM 15 by comparison with N_{TR}. Moreover, x-ray quanta succeeding one another closely in time can be detected in an energy-selective fashion without the need to await the recharging time of the first group 22 of the detection cells 16.

Particularly in the case of small detection cells 16, the energy-selective detection of the x-radiation 8 can be improved by raising the geometric filling factor. To this end, the SiPM 15 can with particular advantage be designed as a backlit SiPM. As against the front-lit SiPMs 15 previously described, such SiPMs exhibit an inverse design with a clearly enlarged active area. Consequently, backlit SiPMs are particularly suitable for x-ray computed tomography applications. A further advantage of backlit SiPMs resides in the possibility of making contact more easily.

N_{trig} is below 10% for the examples #4 and #5 specified in the table. Particularly under these preconditions, it is possible to achieve, for the boundary conditions given in x-ray computed tomography, that a sufficient number of detection cells 16 are still available for immediately sequential x-ray quanta. This means that even the energy of the second x-ray quantum following immediately upon the first x-ray quantum can be detected with sufficient accuracy. In other words: it is possible to avoid considerable impairment of the accuracy of the energy-selective determination due to the temporal vicinity of the impinging x-ray quanta. For example, it is possible to assume for N_{trig}<10% that the error with which energy is determined is likewise under 10%, something which is entirely acceptable. The corresponding statement also holds for the quantitative detection of the x-ray quanta. Owing to the comparatively high number of detection cells 16, the quantum flux rates usually occurring in x-ray computed tomography can be quantitatively detected with sufficient accuracy without considerable saturation-induced falsifications.

In accordance with a further aspect, it can be advantageous when the radiation detector module 9 or the SiPMs 15 and, in particular, also the radiation detector 7, can be operated in a number of operating modes.

To this end, the radiation detector module 9 can, for example, be designed in such a way that at least one parameter of the SiPMs 15 that is essential for detecting the x-radiation 8 in a quantitative and/or energy-selective fashion can be set.

The number of the photons produced per x-ray quantum in the scintillation material, inter alia, comes into consideration for the parameter. The setting can be performed, for example, by a defined, external influence of the conversion efficiency of the scintillation elements 10.

Furthermore, it is possible that only a defined proportion of the triggerable photons produced by the scintillation elements 10 reach the detection cells 16 or photodiodes 17. A possibility here is, for example, an intermediate layer that is provided between the scintillation elements 10 and the detection cells 16 and has an absorption coefficient for the photons which can be set in a defined fashion. Consequently, it is possible to avoid saturation effects associated with high quantum flux rates, for example owing to raising of the absorption coefficient. Owing to the defined setting of the photons produced and/or that can be detected, it is possible to infer the actual number of photons so that the x-radiation 8 can nevertheless be detected accurately.

Furthermore, it is possible to configure the number of sub-photon detection units to be variable. It is possible to this end, for example, to provide that individual sub-photondetection units can be combined with one another or that combinations of the same can be preserved. The advantage resulting therefrom resides in the fact that the counting rate capability of the radiation detector module 9 can be adapted to respective conditions, in particular to quantum flux rates respectively to be expected.

Moreover, it is possible for the PDE to be set and/or varied in a defined fashion. In the case of an SiPM 15 with a pitch of 10 μm and a PDE of 50%, an energy resolution of approximately 11.1% at 30 keV, and of approximately 5.5% at 120 keV can be used as basis. With this comparatively high PDE of 50%, N_{trig} already lies relatively high, at approximately 16.5%, with an energy of 120 keV. However, this comparatively high PDE can be advantageous for certain examinations in x-ray computed tomography. For example, a particularly good energy resolution can be achieved thereby at relatively low quantum flux rates. For higher x-ray flux rates, the PDE can advantageously be varied toward lower values such that the number of the triggered detection cells 16 is reduced, and the counting rate capability is raised. The variation in the PDE can, for example, result from adapting the bias voltage.

The above-described operating mode can be seen within the sense of the invention as a refinement of the previously described first operating mode.

Moreover, the radiation detector module 9 can also be designed in such a way that a second operating mode is provided for detecting the x-radiation 8 in an integrating fashion. It is possible to this end, for example, to switch over an evaluation electronics, or to apply other suitable devices/methods, in such a way that the output signals can be detected in an integrating fashion instead of in the quantitative and/or energy-selective manner. This is advantageous chiefly when the aim is to detect quantum flux rates for which the counting rate capabilities are insufficient. Alternatively, it is also possible for a simultaneous quantitative and/or energy-selective as well as integrating detection of the output signals to take place.

In the first operating mode, it is possible, by way of example, to use a quantum counting first evaluation electronics that evaluates the output signal in an energy-selective manner in the form of individual pulses. In the second operating mode, it is possible to use a charge integrating second evaluation electronics that, for example on the basis of the output signals, integrates or measures the charge generated by x-ray quanta within a prescribed time interval. The switchover between the first and second operating modes can be performed by an electronic switch that connects signal output contacts, or signal lines for the first operating mode to the first evaluation electronics, and connects them to the second evaluation electronics for the second operating mode. The first and second operating electronics can also be designed as a single evaluation electronics having different evaluation modes, it being possible for switching over between the first operating mode and the second operating mode to comprise switching over between the evaluation mode of the evaluation electronics.

Alternatively, the output signal can simultaneously be fed to the first and second readout electronics, that is to say the first and second readout electronics are simultaneously operating, something which is to be regarded as within the scope of the embodiments of the invention. A similar statement holds when the first and second evaluation electronics are designed as a single evaluation electronics and are simultaneously operated both in the first and in the second evaluation modes. For example, the output signal can be switched onto two conduction paths so that both electronics receive the output signal at each instant. In the case of this refinement, it is possible optionally to use quantitative results, or counter results, the quantum counting electronics, integration results, that is to say values of the charges determined by the charge integrating electronics, or both of the counting results and the integration result for the purpose of further evaluation, for example for imagining.

A switchover device—not shown—can be provided in order to switch over the radiation detector module 9 from the first operating mode into the second. This opens up the possibility of, for example, switching over—particularly in an automated fashion—as soon as a prescribed limiting value for the quantum flux rate is overshot. Switching over can, for example, be performed whenever a quantum flux rate to be expected would overshoot the counting rate capability required for detecting the x-radiation with sufficient accuracy. As described above for the first operating mode, an essential parameter of the SiPM can also be switched over in the course of switching over from the first into the second operating modes. For example, it is possible to vary the PDE, for example by varying or adapting the bias voltage in accordance with the requirements of the respective operating mode.

The radiation detector module 9 can, furthermore, comprise a third operating mode for detecting gamma radiation. The gamma radiation can be caused, for example, by positron emission events. It is thereby possible to use a single imaging modality to carry out both x-ray computed tomography examinations and positron emission tomography (PET) examinations. By comparison with x-ray computed tomography, in the case of PET comparatively low quantum flux rates occur that can be detected with entirely adequate accuracy by means of an SiPM 15 adapted to x-ray flux rates. If required, one or more parameters for the respective computed tomography, or PET operation can be adapted to the modality. Consequently, the scintillation material can be designed such that both x-ray quanta and gamma quanta are converted with adequate efficiency into light that can be detected by the SiPM. This apart, it is also possible to use different scintillation materials or scintillation layers for x-radiation or gamma radiation.

On the model of the third operating mode, it is also possible, moreover, to provide a fourth operating mode that enables detection of single photon emission events. It is thereby possible to implement an imaging modality that can be operated in the x-ray computed tomography mode as well as in the PET and/or SPECT mode. Here, SPECT stands for Single Photon Emission Computed tomography. An imaging tomography device can thus comprise an x-ray computed tomography device and a PET and/or SPECT device. Corresponding use is made of the radiation detector module 9 in the case of an x-ray computed tomography device, a combined x-ray positron emission tomography device and/or a combined x-ray single photon emission tomography device. The radiation detector can be designed, for example, as a ring detector in the case of one of the abovementioned combined devices.

Owing to the different operating modes, the application spectrum of the respective imaging modality can be substantially widened, and the procurement costs for such combined modalities can be greatly reduced.

Overall, it becomes clear that at least one of the disadvantages according to the prior art are overcome or at least reduced with the aid of at least one embodiment of the invention. At least one embodiment of the invention makes it possible to provide a scintillator photodetection-based radiation detector module or a corresponding radiation detector for x-ray computed tomography that enables x-radiation to be detected in a quantitative and/or energy-selective fashion. A corresponding statement holds for the imaging tomography device(s).

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A radiation detector module for producing a radiation detector for x-ray computed tomography, having a first operating mode for at least one of quantitative and energy-selective detection of x-radiation, the radiation detector module comprising:
a scintillation layer, produced from a scintillation material, to convert the x-radiation into light; and
a photodetection unit, operatively coupled to the scintillation layer, to detect the light, the photodetection unit including a number of silicon photomultipliers.

2. The radiation detector module as claimed in claim 1, wherein the scintillation layer includes a multiplicity of scintillation elements lined up together in matrix fashion.

3. The radiation detector module as claimed in claim 2, wherein neighboring scintillation elements are separated from one another by septa.

4. The radiation detector module as claimed in claim 3, wherein a width of the septa that is given by the spacing of neighboring scintillation elements lies in the range of between 50 micrometers and 350 micrometers.

5. The radiation detector module as claimed in claim 2, wherein each scintillation element is assigned at least one silicon photomultiplier.

6. The radiation detector module as claimed in claim 2, wherein the size of a detection cross section of a scintillation element is smaller than 15 mm².

7. The radiation detector module as claimed in claim 1, wherein the scintillation material has a decay time in the range of a few nanoseconds up to a few tens of nanoseconds.

8. The radiation detector module as claimed in claim 7, wherein the decay time is smaller than 40 ns to 20 ns.

9. The radiation detector module as claimed in claim 1, wherein the scintillation material is selected from the group consisting of: Lu₂SiO₅:(Ce), LaBr₃:(Ce), YAP:Ce, and Lu(Y)AP:Ce.

10. The radiation detector module as claimed in claim 1, wherein the x-radiation has a quantum flux rate in the range of 1 billion per second and square millimeter.

11. The radiation detector module as claimed in claim 1, wherein x-ray quanta of the x-radiation have an energy of from 30 kiloelectron volts to 120 kiloelectron volts.

12. The radiation detector module as claimed in claim 1, wherein a decay time of an output signal of each silicon photomultiplier lies in the range of a few nanoseconds.

13. The radiation detector module as claimed in claim 1, wherein the silicon photomultiplier includes a multiplicity of detection cells arranged in matrix fashion.

14. The radiation detector module as claimed in claim 13, wherein a recharging time of each detection cell is less than ten nanoseconds.

15. The radiation detector module as claimed in claim 13, wherein a photon detection efficiency of the detection cells is in the range of from 10% to 50.

16. The radiation detector module as claimed in claim 13, wherein the detection cells have a nonlinearity of less than 20%.

17. The radiation detector module as claimed in claim 13, wherein the number of the detection cells of each silicon photomultiplier is greater by at least a factor of approximately two than the number of the photons in the light that are generatable by an x-ray quantum of the x-radiation and are suitable for triggering detection cells.

18. The radiation detector module as claimed in claim 13, wherein bias contacts of the detection cells of each silicon photomultiplier in each case make contact with a single bias line.

19. The radiation detector module as claimed in claim 13, wherein signal contacts of the detection cells of each silicon photomultiplier in each case make contact with a single signal line.

20. The radiation detector module as claimed in claim 18, wherein at least one of the bias lines and the signal lines are routed, in interspaces between the silicon photomultipliers, to the edge of the radiation detector module.

21. The radiation detector module as claimed in claim 13, wherein at least one of bias contacts and signal contacts of the detection cells of each silicon photomultiplier are connected to a single at least one of a bias line and signal line, which lines are routed to an end face edge of the radiation detector module.

22. The radiation detector module as claimed in claim 13, wherein bias contacts of the detection cells are arranged on an underside or top side of the silicon photomultiplier and are each connected to one, or to a common, bias line.

23. The radiation detector module as claimed in claim 13, wherein the detection cells are arranged in rows and columns, and bias contacts of the detection cells are each connected to at least one line or row of detection cells with a common bias line.

24. The radiation detector module as claimed in claim 13, wherein the detection cells are arranged in rows and columns, and signal output contacts on the detection cells are each connected to at least one line or row with a single common signal line.

25. The radiation detector module as claimed in claim 13, wherein a cell period of the detection cells lies in the range of from 50 micrometers to 25 micrometers.

26. The radiation detector module as claimed in claim 13, wherein the number of detection cells per silicon photomultiplier lies between 1500 and 2500.

27. The radiation detector module as claimed in claim 1, wherein the silicon photomultipliers are designed as a backlit silicon photomultiplier.

28. The radiation detector module as claimed in claim 1, wherein each silicon photomultiplier includes a number of sub-photodetection units.

29. The radiation detector module as claimed in claim 2, wherein each scintillation element has a number of sub-scintillation elements that corresponds to the number of the sub-photodetection units.

30. The radiation detector module as claimed in claim 28, wherein a number of the sub-photodetection units or sub-scintillation elements is given by N×N, in which N is a natural number.

31. The radiation detector module as claimed in claim 30, wherein N is equal to 2, 4 or 5.

32. The radiation detector module as claimed in claim 1, wherein the silicon photomultiplier is provided on a substrate and, wherein at least one of electronic components and circuits provided for processing output signals of the silicon photomultipliers or detection cells are provided on the substrate or integrally with the substrate.

33. The radiation detector module as claimed in claim 1, wherein the module is designed in such a way that at least one parameter of the silicon photomultipliers that is essential for at least one of the quantitative and energy-selective detection of the x-radiation is settable.

34. The radiation detector module as claimed in claim 33, wherein the parameter is selected from the following group: photon detection efficiency, number of the sub-photodetection units, number of the photons generated or that can be acquired per x-ray quantum of the x-radiation in the scintillation material, absorption coefficient for photons in an intermediate layer.

35. The radiation detector module as claimed in claim 33, wherein the parameter is settable by varying the bias voltage.

36. The radiation detector module as claimed in claim 1, comprising a second operating mode for the integrating detection of x-radiation.

37. The radiation detector module as claimed in claim 36, wherein the module is designed in such a way that at least one parameter of the silicon photomultipliers that is essential for at least one of the quantitative and energy-selective detection of the x-radiation is settable and wherein switching over from the first into the second operating mode or from the second into the first operating mode comprises setting at least one essential parameter of the silicon photomultiplier.

38. The radiation detector module as claimed in claim 1, further comprising an evaluation electronics that is connected to signal output contacts of the silicon photomultiplier and has at least two evaluation modes, in which a quantitative, energy-selective detection of x-ray quanta is performed in a first evaluation mode, and an integrating detection of charges generated by x-ray quanta in a prescribed time window is performed in a second evaluation mode.

39. The radiation detector module as claimed in claim 38, wherein the evaluation electronics comprise at least a first and a second evaluation unit, it being possible to operate the first evaluation unit in the first evaluation mode and to operate the second evaluation unit in the second evaluation mode.

40. The radiation detector module as claimed in claim 38, wherein switching over between the first operating mode and the second operating mode comprises switching over the evaluation electronics between a first evaluation mode and a second evaluation mode.

41. The radiation detector module as claimed in claim 38, wherein the evaluation electronics is simultaneously operatable in the first and second evaluation modes.

42. The radiation detector module as claimed in claim 38, wherein the first evaluation mode comprises a quantitative, energy-selective determination of x-ray quanta, and the second evaluation mode comprises an integrating detection of charges generated by x-ray quanta in a prescribed time window.

43. The radiation detector module as claimed in claim 38, further comprising a switchover device for switching over from the first to the second operating mode upon overshooting of a prescribed limiting value for the quantum flux rate of the x-radiation.

44. The radiation detector module as claimed in claim 1, further comprising a third operating mode for detecting gamma radiation.

45. The radiation detector module as claimed in claim 44, wherein the scintillation material is designed in such a way that both x-ray quanta and gamma quanta are converted into light that are detectable by way of the silicon photomultipliers.

46. The radiation detector module as claimed in claim 45, wherein the third operating mode is designed in such a way that positron emission events are detectable.

47. The radiation detector module as claimed in claim 46, further comprising a fourth operating mode for detecting single photon emission events.

48. A radiation detector, comprising a number of radiation detector modules as claimed in claim 47.

49. An imaging tomography device, comprising a radiation detector as claimed in claim 48.

50. The imaging tomography device as claimed in claim 49, comprising an x-ray computed tomography device.

51. The imaging tomography device as claimed in claim 50, further comprising a positron emission tomography device.

52. The imaging tomography device as claimed in claim 50, further comprising a single photon emission tomography device.

53. A method comprising:
using the radiation detector as claimed in claim 48 in an x-ray computed tomography device.

54. A method comprising:
using the radiation detector as claimed in claim 48, in a combined x-ray positron emission tomography device.

55. A method comprising:
using the radiation detector as claimed in claim 48, in a combined x-ray single photon emission tomography device.

56. A method comprising:
using the radiation detector module as claimed in claim 1 to produce a radiation detector for an x-ray computed tomography device.

57. A method comprising:
using the radiation detector module as claimed in claim 46 to produce a radiation detector for an x-ray positron emission tomography device.

58. A method comprising:
using the radiation detector module as claimed in claim 47 to produce a radiation detector for an x-ray single photon emission tomography device.

59. The radiation detector module as claimed in claim 4, wherein a width of the septa that is given by the spacing of neighboring scintillation elements lies in the range of between 80 and 300 micrometers.

60. The radiation detector module as claimed in claim 6, wherein the size of a detection cross section of a scintillation element is smaller than 10 mm².

61. The radiation detector module as claimed in claim 60, wherein the size of a detection cross section of a scintillation element is smaller than 1 mm².

62. The radiation detector module as claimed in claim 13, wherein a cell period of the detection cells is less than 10 micrometers.
